(19) United States
(12) Patent Application Publication
(10) Pub. No.: US 2019/0388063 A1
(43) Pub. Date: Dec. 26, 2019

(54) ULTRASOUND DIAGNOSTIC APPARATUS, ULTRASOUND DIAGNOSTIC METHOD, AND COMPUTER-READABLE RECORDING MEDIUM

(71) Applicant: Konic Minolta, Inc., Tokyo (JP)

(72) Inventors: Toshio OKA, Tokyo (JP); Yusuke TANABE, Tokyo (JP)

(21) Appl. No.: 16/440,673

(22) Filed: Jun. 13, 2019

(30) Foreign Application Priority Data
Jun. 20, 2018 (JP) 2018-116836

(51) Int. Cl. A61B 8/08; A61B 8/00

(52) U.S. Cl. CPC A61B 8/5207; A61B 8/4444; A61B 8/4427; G16H 30/40; A61B 8/5246; A61B 8/4433; A61B 8/4405; A61B 8/461

(57) ABSTRACT

An ultrasound diagnostic apparatus includes: an ultrasound image generation section that generates an ultrasound image from the reception signal obtained by an ultrasound probe; a panorama image generation section that stitches together a plurality of ultrasound images to generate a panorama image, the ultrasound images being successively acquired while the ultrasound probe is moved; a display processing section that causes a display section to display an object indicating information necessary for image diagnosis together with the ultrasound image or the panorama image; an acquisition section that acquires information on a moving speed of the ultrasound probe; and a display attribute setting section that changes a display attribute of a specific object which is at least one of objects in accordance with the information on the moving speed of the ultrasound probe.

### CROSS REFERENCE TO RELATED APPLICATIONS

The entire disclosure of Japanese Patent Application No. 2018-116836 filed on Jun. 20, 2018 is incorporated herein by reference in its entirety.

### BACKGROUND

### Technological Field

The present invention relates to an ultrasound diagnostic apparatus, an ultrasound diagnostic method, and a computer-readable recording medium, and particularly relates to a technique that is useful at the time of stitching together (connecting together) a plurality of ultrasound images to generate a panorama image.

### Description of Related Art

As one of medical image diagnostic apparatuses, there is known an ultrasound diagnostic apparatus that transmits ultrasound to a subject, receives reflected waves of the ultrasound, and performs predetermined signal processing on the reception signal to visualize the shape, the properties, or the dynamic state of the inside of the subject as an ultrasound image. The ultrasound diagnostic apparatus, which makes it possible to acquire an ultrasound image by a simple operation of putting an ultrasound probe on the body surface or inserting the probe into the body, is safe and imposes less burden on the subject.

Some of such ultrasound diagnostic apparatuses are provided with a panorama mode in which successive ultrasound images (B-mode images) are acquired by transmitting and receiving ultrasound while moving the ultrasound probe along the diagnosed site of the subject, and the acquired ultrasound images are stitched together and displayed as one panorama image.

When an ultrasound image is acquired and displayed in a state where the ultrasound probe is fixed, an observable range is small and it is difficult to see which position in the subject is being viewed, thus causing a problem of insufficient objectivity in the image. In contrast, the panorama mode makes it possible to represent the diagnosed site in a broad range and thereby facilitate observing and diagnosing the entire image of the diagnosed site.

In the panorama mode, overlap and correlation between adjacent images are obtained to stitch ultrasound images together. However, when a probe moving operation for acquiring the ultrasound images is too fast, the overlap and correlation are reduced, which might prevent generation of a panorama image with high accuracy. When the probe moving operation is too slow, the overlap increases, leading to acquisition of increased number of images unnecessary for generation of the panorama image, which is inefficient. Therefore, in the panorama mode, it is important to move the ultrasound probe at an appropriate speed and acquire ultrasound images in a proper quantity.

Examples of a technique for assisting the probe moving operation at the time of generating a panorama image include a technique of Japanese Patent Application Laid-Open No. 2014-100270. Japanese Patent Application Laid-Open No. 2014-100270 discloses that at the time of acquiring a panorama image, a guide is displayed so as to make the moving speed of the ultrasound probe appropriate.

In the method disclosed in Japanese Patent Application Laid-Open No. 2014-100270, a horizontal bar-like figure displayed together with the panorama image extends at a constant speed from the start of panorama shooting, or a numerical value displayed on a screen increases, to assist the probe moving operation. A user can confirm the progress degree of the shooting from the horizontal bar-like figure or the numerical value and can move the probe in accordance with the progress degree of the shooting. However, in the technique of Japanese Patent Application Laid-Open No. 2014-100270 the user needs to perform complicated operation and determination of making visual confirmation of the progress degree of the shooting, further making visual confirmation of the moving quantity of the probe with respect to the entire filmed portion, and determining whether or not those two progress degrees agree. On the other hand, since the user performs the shooting while confirming the ultrasound image displayed on the ultrasound diagnostic apparatus at the time of shooting the ultrasound image, it is difficult for the user to confirm and determine the progress degrees by the method disclosed in Japanese Patent Application Laid-Open No. 2014-100270.

### SUMMARY

An object of the present invention is to provide an ultrasound diagnostic apparatus, an ultrasound diagnostic method, and a computer-readable recording medium which can assist a moving operation of an ultrasound probe at the time of generating a panorama image.

To achieve at least one of the abovementioned objects, according to an aspect of the present invention, an ultrasound diagnostic apparatus reflecting one aspect of the present invention is an apparatus that drives an ultrasound probe so as to transmit ultrasound toward a subject and receives a reception signal based on reflected waves reflected in the subject from the ultrasound probe, to generate and display an ultrasound image, the apparatus comprising a hardware processor that:

generates an ultrasound image from the reception signal obtained by the ultrasound probe;

stitches together a plurality of the ultrasound images to generate a panorama image, the plurality of ultrasound images being successively acquired while the ultrasound probe is moved;

causes a display to display an object indicating information necessary for image diagnosis together with the ultrasound image or the panorama image;

acquires information on a moving speed of the ultrasound probe; and

changes a display attribute of at least one of the objects in accordance with the information on the moving speed of the ultrasound probe.

To achieve at least one of the abovementioned objects, according to an aspect of the present invention, an ultrasound diagnostic method reflecting one aspect of the present invention is a method for driving an ultrasound probe so as to transmit ultrasound toward a subject and receiving a reception signal based on reflected waves reflected in the subject from the ultrasound probe, to generate and display an ultrasound image, the method comprising:

generating an ultrasound image from the reception signal obtained by the ultrasound probe;

stitching together a plurality of the ultrasound images to generate a panorama image, the ultrasound images being successively acquired while the ultrasound probe is moved;

causing a display to display an object that indicates information necessary for image diagnosis together with the ultrasound image or the panorama image;

acquiring information on a moving speed of the ultrasound probe; and

changing a display attribute of at least one of the objects in accordance with the information on the moving speed of the ultrasound probe.

To achieve at least one of the abovementioned objects, according to an aspect of the present invention, a non-transitory computer-readable storage medium reflecting one aspect of the present invention is a medium storing a program causing a computer to execute processing, the computer being of an ultrasound diagnostic apparatus that drives an ultrasound probe so as to transmit ultrasound toward a subject and receives a reception signal based on reflected waves reflected in the subject from the ultrasound probe, to generate and display an ultrasound image, the processing to be executed by the computer, comprising:

generating an ultrasound image from the reception signal obtained by the ultrasound probe;

stitching together a plurality of the ultrasound images to generate a panorama image, the ultrasound images being successively acquired while the ultrasound probe is moved;

causing a display to display an object that indicates information necessary for image diagnosis together with the ultrasound image or the panorama image;

acquiring information on a moving speed of the ultrasound probe; and

changing a display attribute of at least one of the objects in accordance with the information on the moving speed of the ultrasound probe.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 illustrates an appearance of an ultrasound diagnostic apparatus according to an embodiment;

FIG. 2 illustrates a configuration of an ultrasound probe;

FIG. 3 is a block diagram illustrating a main part of a control system of the ultrasound diagnostic apparatus;

FIG. 4 illustrates an example of a screen displayed on a display section;

FIG. 5 illustrates an example of an attribute table;

FIG. 6 is a flowchart illustrating an example of panorama image generation processing; and

FIGS. 7A to 7C illustrate examples of a panorama image generation process.

### DETAILED DESCRIPTION OF EMBODIMENTS

Hereinafter, one or more embodiments of the present invention will be described with reference to the drawings. However, the scope of the invention is not limited to the disclosed embodiments.

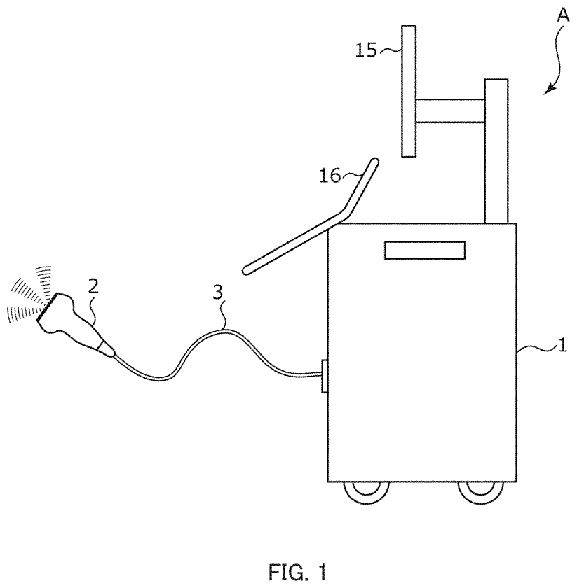

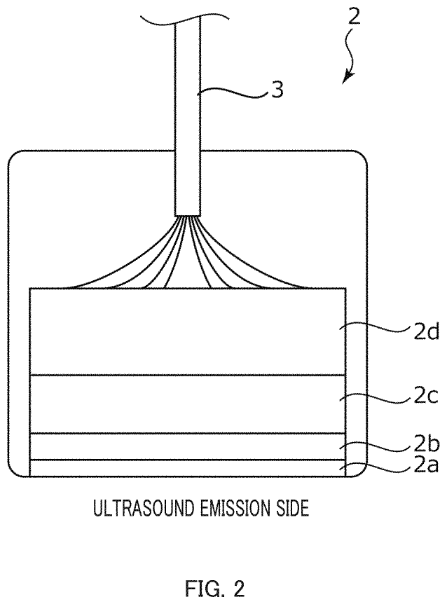

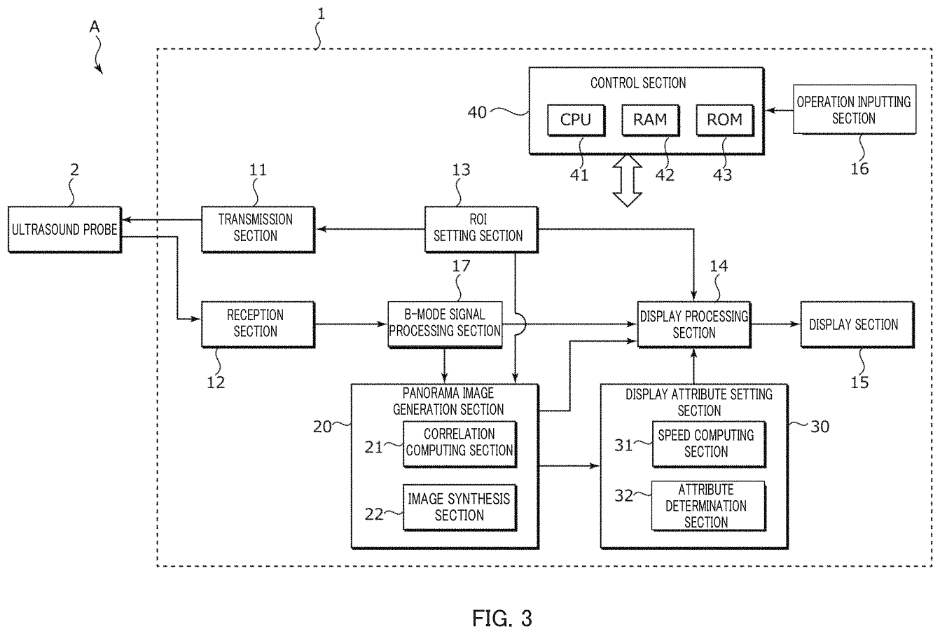

FIG. 1 illustrates an appearance of ultrasound diagnostic apparatus A according to an embodiment of the present invention. FIG. 2 illustrates a configuration of ultrasound probe 2. FIG. 3 is a block diagram illustrating a main part of a control system of ultrasound diagnostic apparatus A.

As illustrated in FIG. 1, ultrasound diagnostic apparatus A is provided with ultrasound diagnostic apparatus main unit 1 and ultrasound probe 2. Ultrasound diagnostic apparatus main unit 1 and ultrasound probe 2 are connected to each other through cable 3. Note that ultrasound probe 2 may be connected to ultrasound diagnostic apparatus main unit 1 through wireless communication.

Ultrasound diagnostic apparatus A is used to visualize the shape, properties, or the dynamic state of the inside a subject as an ultrasound image and perform image diagnosis. As diagnostic modes, ultrasound diagnostic apparatus A has, for example, a mode (hereinafter referred to as “B-mode”) for displaying only a B-mode image and a mode (hereinafter referred to as “panorama mode”) for displaying a panorama image formed by stitching together (connecting together) B-mode images having been successively acquired with ultrasound probe 2 on the move. Generally, the panorama image is generated by cutting out a high-quality portion set as a region of interest (ROI) from the B-mode image and stitching the cut-out high-quality portions together. In the panorama mode, for example, a user manually moves ultrasound probe 2 in a scanning direction (longitudinal direction).

Ultrasound probe 2 transmits ultrasound to the subject and receives an ultrasound echo reflected by the subject. Ultrasound probe 2 then converts the ultrasound echo into a reception signal and transmits the converted signal to ultrasound diagnostic apparatus main unit 1. An arbitrary electronic scanning probe, such as a convex probe, a linear probe, or a sector probe, can be applied to ultrasound probe 2.

As illustrated in FIG. 2, ultrasound probe 2 has acoustic lens 2a, acoustic matching layer 2b, transducer array 2c, and backing member 2d in order from the ultrasound emission side. Note that the protective layer may be disposed on the surface (ultrasound emission surface) of acoustic lens 2a.

Acoustic lens 2a is a lens for converging ultrasound in a slicing direction and has, for example, a semi-cylindrical shape with its center raised in the slicing direction.

Acoustic matching layer 2b is an intermediate material for allowing an efficient entry of the ultrasound into the subject and matches acoustic impedances of a transducer (not illustrated) and a shooting object.

Transducer array 2c is made up of a plurality of strip-like transducers (not illustrated) arranged on a single row or multiple rows in the scanning direction.

Backing member 2d attenuates unnecessary vibration generated in transducer array 2c.

With ultrasound probe 2, it is possible to obtain a beam profile for ultrasound that is converged in the slicing direction. Further, by switching the transducer to be driven, it is also possible to converge the ultrasound in the scanning direction (so-called electronic scanning system).

Ultrasound diagnostic apparatus main unit 1 uses the reception signal from ultrasound probe 2 to visualize the internal state of the subject as an ultrasound image. As illustrated in FIG. 3, ultrasound diagnostic apparatus main unit 1 is provided with transmission section 11, reception section 12, ROI setting section 13, display processing section 14, display section 15, operation inputting section 16, B-mode signal processing section 17, panorama image generation section 20, display attribute setting section 30, control section 40, and the like

Transmission section 11, reception section 12, ROI setting section 13, display processing section 14, B-mode signal processing section 17, panorama image generation section 20, and display attribute setting section 30 are formed of at least one dedicated hardware (electronic circuit) corresponding to each processing, such as a digital signal processor (DSP), an application specific integrated circuit (ASIC), and a programmable logic device (PLD).

Control section 40 has central processing unit (CPU) 41 as a computing/control apparatus, read only memory (ROM) 43 and random access memory (RAM) 42 as a main storage apparatus, and the like. Basic programs and basic setting data are stored into a ROM 43. CPU 41 reads a program corresponding to a processing content from ROM 43, loads the read program into RAM 42, and executes the loaded program to perform concentrated control on the operation of each functional block (transmission section 11, reception section 12, ROI setting section 13, display processing section 14, display section 15, B-mode signal processing section 17, panorama image generation section 20, and display attribute setting section 30) of ultrasound diagnostic apparatus main unit 1.

In the present embodiment, the function of each functional block is realized by cooperation of control section 40 and each hardware constituting the functional block. Note that a part or all of the function of each functional block may be realized by control section 40 executing the program.

In accordance with the instruction of control section 40, transmission section 11 generates a transmission signal (drive signal) and outputs the generated signal to ultrasound probe 2. Although not illustrated, transmission section 11 has, for example, a clock generation circuit, a pulse generation circuit, a pulse width setting section, and a delay circuit.

The clock generation circuit generates a clock signal that determines the transmission timing and a transmission frequency for a pulse signal. The pulse generation circuit generates a rectangular wave pulse of a bipolar type with a voltage magnitude previously set in a predetermined cycle. The pulse width setting section sets the pulse width of the rectangular wave pulse output from the pulse generation circuit. The rectangular wave pulse generated in the pulse generation circuit is divided into different wiring paths for individual transducers of ultrasound probe 2 after or before being input into the pulse width setting section. The delay circuit delays the generated rectangular wave pulse in accordance with the transmission timing for each transducer and outputs the delayed pulse to ultrasound probe 2.

In accordance with the instruction of control section 40, reception section 12 receives the reception signal from ultrasound probe 2 and outputs the reception signal to B-mode signal processing section 17. Although not illustrated, reception section 12 has, for example, an amplifier, an analog-to-digital (A/D) conversion circuit, and a phasing addition circuit.

The amplifier amplifies the reception signal corresponding to the ultrasound received by each transducer of ultrasound probe 2 at a previously set predetermined amplification factor. The A/D conversion circuit converts the amplified reception signal into digital data at a predetermined sampling frequency. The phasing addition circuit adds a delay time to the reception signal subjected to the A/D conversion for each wiring path corresponding to the transducer to regulate a time phase and adds the regulated phase (phasing addition).

ROI setting section 13 sets a region of interest (ROI) in the B-mode image in accordance with the instruction of control section 40. The region of interest refers to a diagnosis target portion in the B-mode image acquired by ultrasound probe 2 and is set so as to include the diagnosis target portion (e.g., a blood flowing part). ROI setting section 13 sets, for example, a region having been set on the B-mode image by the operation of operation inputting section 16, as the region of interest. The region of interest is displayed with a ROI frame on the B-mode image. In the case of the panorama generation processing, the region of interest is used as a target indicating a target region for generating a panorama image.

In accordance with the instruction of control section 40, display processing section 14 converts image data from each of B-mode signal processing section 17 and panorama image generation section 20 into a display signal corresponding to display section 15 and outputs the converted signal to cause display section 15 to display the B-mode image or the panorama image. For example, at the time of selecting the B-mode, display processing section 14 converts the B-mode image data from B-mode signal processing section 17 into a display signal and outputs the converted signal to display section 15. At the time of selecting the panorama mode, display processing section 14 converts the panorama image data from panorama image generation section 20 into a display signal and outputs the converted signal to display section 15. Display processing section 14 superimposes the ROI frame on the B-mode image or the panorama image in accordance with the setting of the region of interest by ROI setting section 13.

Further, display processing section 14 causes display section 15 to display objects each indicating information necessary for the image diagnosis, together with the B-mode image or the panorama image. An initial value of a display attribute of each object is stored in display processing section 14, for example. In the panorama mode, display processing section 14 controls a display attribute of a specific object based on attribute information from display attribute setting section 30.

Here, examples of the information necessary for the image diagnosis include diagnosis information, an image parameter, a user interface in the case of display section 15 functioning as operation inputting section 16, and a region of interest. The objects include a figure, a character string, an operation button, and the ROI frame. The display attributes of the object include at least one of a display color, a display thickness, a display status (lighting, blinking, changes in display brightness and display color, etc.), and a line type (dotted line, broken line, double line, etc.) of the object.

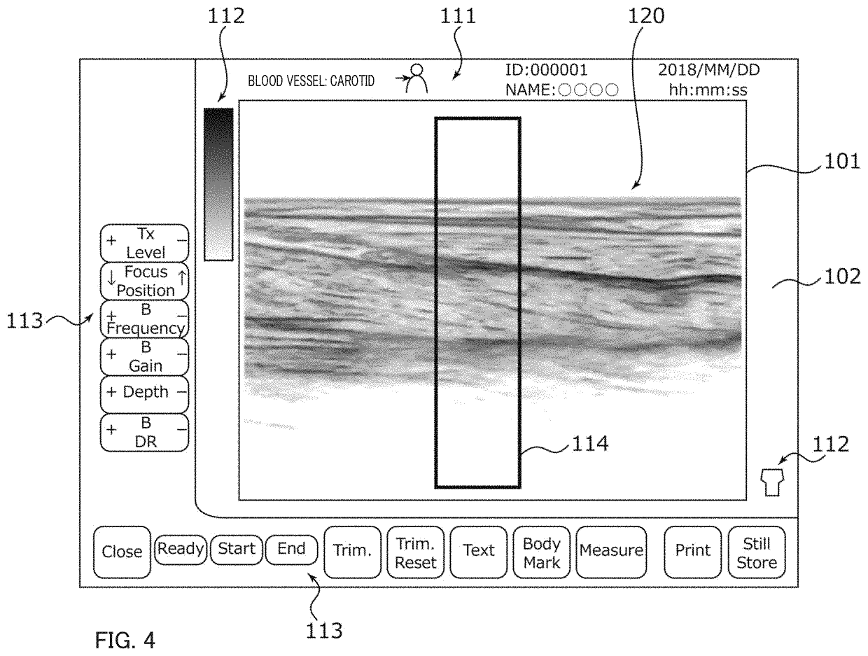

Display section 15 is formed of, for example, a liquid crystal display, an organic electro-luminescence (EL) display, a cathode-ray tube (CRT) display, or the like. In accordance with the instruction of control section 40, display section 15 displays an image based on the display signal from display processing section 14. Display section 15 displays ultrasound image 120 and objects 111 to 114 indicating the information necessary for the image diagnosis (cf. FIG. 4).

Operation inputting section 16 accepts information on diagnosis, for example. Operation inputting section 16 has, for example, an operation panel having a plurality of input switches, a keyboard, a mouse, and the like. Note that operation inputting section 16 may be formed of a touch panel provided integrally with display section 15. The user can set the region of interest, the diagnosed site, the type of ultrasound probe 2, the diagnostic mode (B-mode or panorama mode), and the like via operation inputting section 16.

In accordance with the instruction of control section 40, B-mode signal processing section 17 performs envelope demodulation processing, logarithm compression processing, or the like on the data received from reception section 12 to adjust a dynamic range and a gain for luminance conversion, thereby generating a B-mode image. The data of the generated B-mode image is output to display processing section 14 and panorama image generation section 20. Note that B-mode signal processing section 17 includes a digital scan converter (DSC) that performs coordinate conversion and pixel interpolation in accordance with the type of ultrasound probe 2.

Panorama image generation section 20 generates a panorama image based on B-mode images from B-mode signal processing section 17. Specifically, panorama image generation section 20 stitches together the region of interests on the B-mode images to generate a panorama image. In the present embodiment, panorama image generation section 20 has correlation computing section 21 and image synthesis section 22. In the following, the portion of the region of interest in the B-mode image, the portion being used for generating a panorama image, will be referred to as a “ROI image.”

Correlation computing section 21 computes correlation information indicating the correlation between two images to be synthesized. The two images to be synthesized are, for example, a panorama image (hereinafter referred to as “intermediate panorama image”) generated by stitching together ROI images acquired from the start of the diagnosis to a certain time point, and a newly acquired ROI image to be synthesized with the intermediate panorama image. The correlation information is represented by, for example, a distance vector between characteristic points having the same content, extracted in the intermediate panorama image and the new ROI image. The computed correlation information is output to display attribute setting section 30. Note that the characteristic points having the same content are extracted by extracting characteristic points regarded as having the same content in the intermediate panorama image and the new ROI image. Then, the two pieces of data extracted as the characteristic points having the same content are determined as information on the same point of observation.

Note that the correlation information is also used when speed computing section 31, described later, computes a probe moving speed. That is, the correlation information can also be said to be information on the probe moving speed. In the present embodiment, correlation computing section 21 functions as an “acquisition section” and acquires information on the moving speed of ultrasound probe 2.

Image synthesis section 22 synthesizes the intermediate panorama image and the new ROI image so that the extracted characteristic points having the same content match, namely, the two extracted characteristic points having the same content are the same point of observation. In the synthesis, various modification processing may be performed on the ROI image as necessary. The data of the newly generated intermediate panorama image is output to display processing section 14. Note that a known technique can be applied to the method for generating a panorama image.

Display attribute setting section 30 changes the display attribute of at least one of objects 111 to 114 (e.g., ROI frame 114, hereinafter referred to as “specific object”) in accordance with the moving speed of ultrasound probe 2 (hereinafter referred to as “probe moving speed”) at the time when the panorama image is generated. In the present embodiment, display attribute setting section 30 has speed computing section 31 and attribute determination section 32.

Speed computing section 31 computes the probe moving speed based on the correlation information from panorama image generation section 20 (correlation computing section 21). Specifically, the movement distance of ultrasound probe 2 is obtained from the distance vector contained in the correlation information and the movement time of ultrasound probe 2 is obtained from a frame rate in the panorama mode, so that the probe moving speed is calculated by “movement distance/movement time.” Further, in consideration of rotation of ultrasound probe 2, an arbitrary point (e.g., the apex of the ROI, etc.) on the image centered at the characteristic point may also be calculated, and from a result of synthesizing the calculated movement distance with the movement distance of ultrasound probe 2, calculated from the correlation information, and the movement time, the probe moving speed may be calculated.

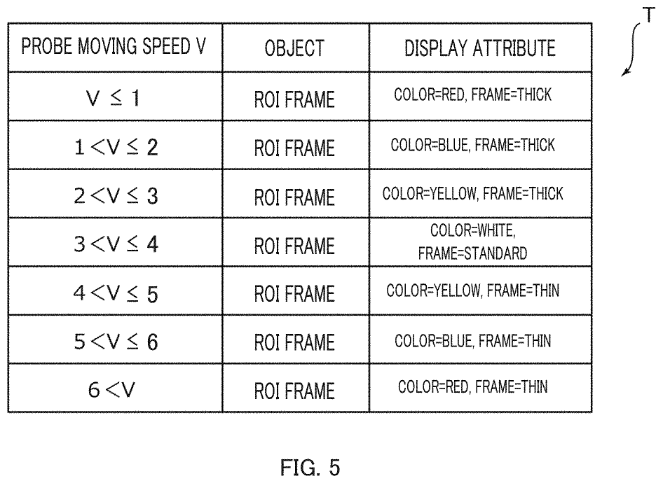

Attribute determination section 32 determines display attributes of the specific object based on the calculated probe moving speed. For example, attribute determination section 32 determines the display attributes of the specific object based on the magnitude of the probe moving speed with respect to the optimum speed (whether the probe moving speed is faster or slower than the optimum speed). Attribute determination section 32 preferably determines the display attributes of the specific object based on a deviation between the probe moving speed and the optimum speed. In the present embodiment, attribute determination section 32 determines the display attributes of the specific object in accordance with the probe moving speed with reference to attribute table T (cf. FIG. 5) in which the display color and display thickness are set in stages. The determined display attributes are output to display processing section 14 as the attribute information of the specific object.

FIG. 4 illustrates an example of a screen displayed on display section 15.

As illustrated in FIG. 4, the screen of display section 15 is divided into first screen region 101 disposed around the center and second screen region 102 outside the first screen region. In first screen region 101, ultrasound image (B-mode image or panorama image) 120 is displayed. In second screen region 102, objects 111 to 114 indicating the information necessary for the image diagnosis are displayed.

Object 111 displayed in an upper part of second screen region 102 is diagnosis information containing patient information, the current date and time, annotation, a body mark (diagnosed site), and the like. In second screen region 102, objects 112 displayed on both sides of first screen region 101 are image parameters such as a focus, a depth, a strength, a probe type, and the like of ultrasound. Objects 113 displayed on the left side of and below second screen region 102 are user interfaces for performing various operations such as image parameter setting. Object 114 in a rectangular shape, superimposed on ultrasound image 120, is the ROI frame (hereinafter referred to as “ROI frame 114”).

In the present embodiment, among objects 111 to 114, ROI frame 114 is used as the specific object and displayed with the display attributes corresponding to the probe moving speed.

FIG. 5 is an attribute table indicating the display attributes of the specific object corresponding to the probe moving speed. FIG. 5 illustrates a case in which the specific object is ROI frame 114. Note that in FIG. 5, a probe moving speed V is represented by a normalized numerical value.

According to attribute table T illustrated in FIG. 5, the probe moving speed V in the case of 3<V≤4 is the optimum speed suitable for generation of a panorama image. In this case, ROI frame 114 is displayed in white with a standard thickness.

When the probe moving speed V is slower than the optimum speed (V≤3), the thickness of ROI frame 114 is set greater than the standard thickness, and the display color of ROI frame 114 changes from white to yellow, blue, and red in accordance with a deviation of the probe moving speed V from the optimum speed.

When the probe moving speed V is faster than the optimum speed (4<V), the thickness of ROI frame 114 is set smaller than the standard thickness, and the display color of ROI frame 114 changes from white to yellow, blue, and red in accordance with the deviation of the probe moving speed V from the optimum speed.

That is, when the moving speed of ultrasound probe 2 is not the optimum speed, the display attribute of ROI frame 114 is changed.

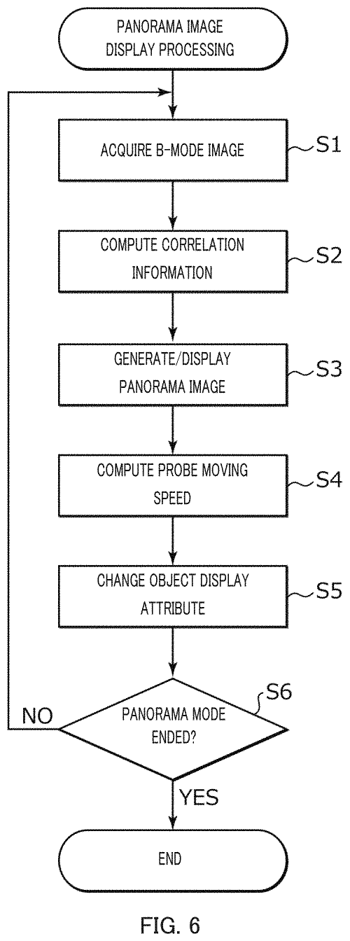

FIG. 6 is a flowchart illustrating an example of the ultrasound diagnostic processing. This processing is realized by, for example, CPU 41 executing a predetermined program stored in ROM 43 in association with the selection of the panorama mode in ultrasound diagnostic apparatus A. The panorama mode is selected by, for example, the selection of the diagnostic mode in operation inputting section 16

In step S1 of FIG. 6, control section 40 controls transmission section 11, reception section 12, and B-mode signal processing section 17 so as to transmit/receive ultrasound via ultrasound probe 2 and acquire a B-mode image.

The data of the generated B-mode image is output to panorama image generation section 20.

In step S2, control section 40 controls panorama image generation section 20 (correlation computing section 21) so as to compare a newly acquired ROI image with an intermediate panorama image and compute correlation information. The ROI image is cut out from the B-mode image acquired in step S1. The computed correlation information (distance vector) is output to display attribute setting section 30.

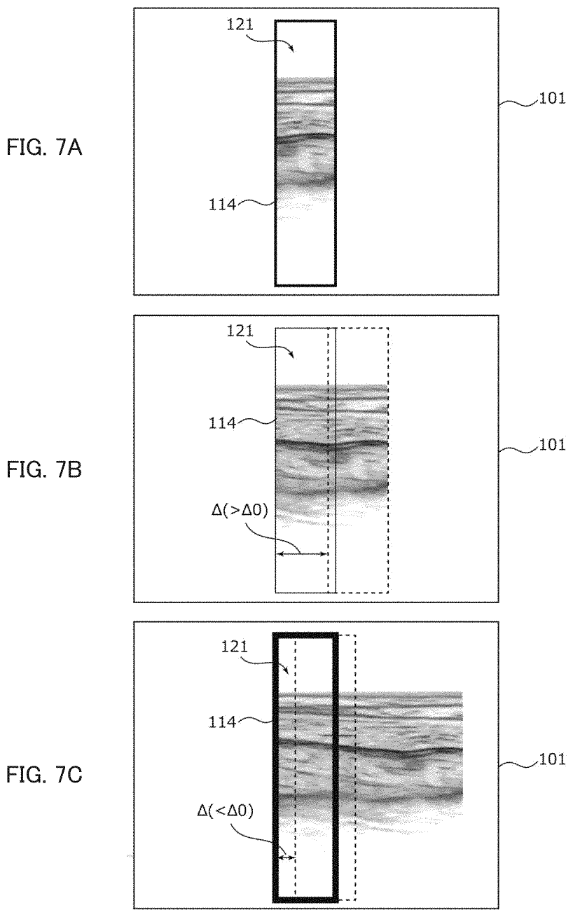

In step S3, control section 40 controls panorama image generation section 20 (image synthesis section 22) and display processing section 14 so as to synthesize the newly acquired ROI image and the intermediate panorama image and display the synthesized image. A panorama image having been generated up to that point is displayed on display section 15 (cf. FIGS. 7B and 7C).

In step S4, control section 40 controls display attribute setting section 30 (speed computing section 31) so as to calculate a probe moving speed.

In step S5, control section 40 controls display attribute setting section 30 (attribute determination section 32) and display processing section 14 so as to change the display attribute of ROI frame 114 in accordance with the probe moving speed (cf. FIGS. 7B and 7C). The user can intuitively see the appropriateness of the current probe moving speed from the display attributes (display color and display thickness) of ROI frame 114. Then, by moving Ultrasound probe 2 so that the display attributes of ROI frame 114 indicate the optimum speed, it is possible to efficiently generate a panorama image with high accuracy.

In step S6, control section 40 determines whether or not a panorama mode ending operation has been input. When the panorama mode ending operation has been input (“YES” in step S6), the processing is ended. When the panorama mode ending operation has not been input (“NO” in step S6), the processing shifts to step S1. By repetition of steps S1 to S6, the panorama image is sequentially expanded, leading to the eventual generation of the panorama image in a broad range.

Note that the first acquired B-mode image is not subjected to the processing of steps S2 to S5, and the panorama image (ROI image 121) is simply displayed in ROI frame 114 (cf. FIG. 7A). Further, in step S5, when the display attributes of ROI frame 114 determined by attribute determination section 32 are the same as the initial values (display color: white, display thickness: standard), no change is made on the display attribute of ROI frame 114.

FIGS. 7A to 7C illustrate examples of the panorama image generation process. In FIGS. 7A to 7C, image 121 displayed in ROI frame 114 is the latest ROI image.

FIG. 7A illustrates a state in which the first B-mode image has been acquired. In the state illustrated in FIG. 7A, no determination is made on the probe moving speed, and hence ROI frame 114 is displayed in accordance with its initial display attributes (white, standard thickness).

FIGS. 7B and 7C illustrate cases in which ultrasound probe 2 has been moved to the left side. FIG. 7B illustrates a case in which the probe moving speed has become faster than the optimum speed during the generation of the panorama image. FIG. 7C illustrates a case in which the probe moving speed has become slower than the optimum speed during the generation of the panorama image. In each of FIGS. 7B and 7C, a region of a ROI image acquired by immediately previous transmission/reception of ultrasound is indicated by a dotted line.

As illustrated in FIGS. 7B and 7C, when ultrasound probe 2 is moved to the left side, a ROI image on the left side of the previously acquired ROI image (the region surrounded by the dotted line in each of FIGS. 7B and 7C) is acquired, and the panorama image is sequentially generated and displayed. In FIGS. 7B and 7C, with ROI frame 114 being fixed, the panorama image extends to the right side. When the panorama image reaches the right end of first screen region 101, the panorama image extends to the left side while ROI frame 114 moves to the left side.

As illustrated in FIG. 7B, when a difference Δ from the previous time is greater than a difference Δ0 from the optimum speed and the probe moving speed is faster than the optimum speed, ROI frame 114 is displayed with a thickness smaller than the standard thickness. Although not displayed in FIG. 7B, ROI frame 114 is displayed with a display color corresponding to the deviation between the probe moving speed and the optimum speed. For example, according to attribute table T illustrated in FIG. 5, when the probe moving speed V is 6<V, ROI frame 114 is displayed with a red thin line.

On the other hand, as illustrated in FIG. 7C, when the difference Δ from the previous time is smaller than the difference Δ0 from the optimum speed and the probe moving speed is slower than the optimum speed, ROI frame 114 is displayed with a thickness greater than the standard thickness. Although not displayed in FIG. 7C, ROI frame 114 is displayed with a display color corresponding to the deviation between the probe moving speed and the optimum speed. For example, according to attribute table T illustrated in FIG. 5, when the probe moving speed V is 1<V≤2, ROI frame 114 is displayed with a blue thick line.

As described above, ultrasound diagnostic apparatus A according to the embodiment drives ultrasound probe 2 so as to transmit ultrasound toward a subject and receives a reception signal based on reflected waves reflected in the subject from ultrasound probe 2, to generate and display an ultrasound image. Ultrasound diagnostic apparatus A is provided with: B-mode signal processing section 17 (ultrasound image generation section) that generates a B-mode image (ultrasound image) from the reception signal obtained by ultrasound probe 2; panorama image generation section 20 that stitches together a plurality of ultrasound images, successively acquired while ultrasound probe 2 is moved, to generate a panorama image; display processing section 14 that causes display section 15 to display objects 111 to 114 indicating information necessary for image diagnosis together with the B-mode image or the panorama image; correlation computing section 21 (acquisition section) that acquires information on a moving speed of ultrasound probe 2; and display attribute setting section 30 that changes a display attribute of a specific object (at least one of objects 111 to 114, e.g., ROI frame 114) in accordance with the information on the moving speed of ultrasound probe 2.

Further, the ultrasound diagnostic method of the present embodiment is a method of driving ultrasound probe 2 so as to transmit ultrasound toward a subject and receiving a reception signal based on reflected waves reflected in the subject from ultrasound probe 2, to generate and display an ultrasound image. The ultrasound diagnostic method includes: generating a B-mode image (ultrasound image) from the reception signal obtained by ultrasound probe 2 (step S1 in FIG. 6); stitching together a plurality of ultrasound images, successively acquired while ultrasound probe 2 is moved, to generate a panorama image (steps S2 and S3 in FIG. 6); causing display section 15 to display objects 111 to 114 that indicates information necessary for image diagnosis together with the B-mode image or the panorama image (steps S4 and S5 in FIG. 6); acquiring information on a moving speed of ultrasound probe 2 (step S2 in FIG. 6); and changing a display attribute of a specific object (at least one of objects 111 to 114, e.g., ROI frame 114) in accordance with the information on the moving speed of ultrasound probe 2 (steps S4 and S5 in FIG. 6).

Moreover, the program according to the present embodiment is a program for causing control section 40 (computer) of ultrasound diagnostic apparatus A to execute the following processing, the ultrasound diagnostic apparatus A driving ultrasound probe 2 so as to transmit ultrasound toward a subject and receiving a reception signal based on reflected waves reflected in the subject from ultrasound probe 2, to generate and display an ultrasound image: generating a B-mode image (ultrasound image) from the reception signal obtained by ultrasound probe 2 (step S1 in FIG. 6); stitching together a plurality of ultrasound images, successively acquired while ultrasound probe 2 is moved, to generate a panorama image (steps S2 and S3 in FIG. 6); causing display section 15 to display objects 111 to 114 that indicates information necessary for image diagnosis together with the B-mode image or the panorama image (steps S4 and S5 in FIG. 6); acquiring information on a moving speed of ultrasound probe 2 (step S2 in FIG. 6); and changing a display attribute of a specific object (at least one of objects 111 to 114, e.g., ROI frame 114) in accordance with the information on the moving speed of ultrasound probe 2 (steps S4 and S5 in FIG. 6).

This program is provided via, for example, a computer-readable portable recording medium (including an optical disc, a photo-magnetic disc, and a memory card) in which the program is stored. Further, this program can also be provided by, for example, download from a server, which holds the program, through a network.

According to ultrasound diagnostic apparatus A, the ultrasound diagnostic method, and the program according to the embodiment, it is possible to cause the user to intuitively see the probe moving speed, and thus possible to assist the movement operation of ultrasound probe 2 at the time of generating a panorama image. The user may only move ultrasound probe 2 so that the probe moving speed becomes the optimum speed. Since objects 111 to 114 are basic display elements indicating information necessary for the image diagnosis, there is no need for newly adding a display element so as to display assist information suggesting the probe moving speed, and the visibility of the panorama image under generation is not inhibited.

In ultrasound diagnostic apparatus A, display attribute setting section 30 changes the display attribute of at least one of objects 111 to 114 (specific object) based on the magnitude of the moving speed of ultrasound probe 2 with respect to a predetermined optimum speed.

It is thereby possible to deal with both a case where the probe moving speed is excessively faster than the optimum speed and a case where the probe moving speed is excessively slower than the optimum speed. This enables efficient generation of a panorama image with high accuracy.

In ultrasound diagnostic apparatus A, display attribute setting section 30 changes the display attribute of the specific object (e.g., ROI frame 114) based on a deviation between the moving speed of ultrasound probe 2 and the optimum speed.

This enables the user to see the degree of deviation of the probe moving speed from the optimum speed, so that the user can easily adjust the probe moving speed.

In ultrasound diagnostic apparatus A, the display attributes of the specific object (ROI frame 114) includes at least one of a display color, a display thickness, a display status (lighting, blinking, changes in display brightness and display color, etc.), and a line type (dotted line, broken line, double line, etc.).

This can facilitate the user to visually see the appropriateness of the probe moving speed.

In ultrasound diagnostic apparatus A, display attribute setting section 30 changes the display attribute of the specific object (e.g., ROI frame 114) in accordance with the moving speed of ultrasound probe 2 with reference to display attribute table Tin which at least one of the display color and the display thickness is set in stages.

This reduces a load on control section 40 when the display attribute of the specific object is changed, so that the probe moving speed can be promptly reflected in the display of the specific object.

In ultrasound diagnostic apparatus A, the specific object is preferably ROI frame 114 indicating a target region at the time of generating the panorama image.

This enables the user to easily see the probe moving speed while confirming the panorama image being generated without shifting the point of view and thereby acquire an appropriate image.

In ultrasound diagnostic apparatus A, display attribute setting section 30 changes the display attribute of the specific object (e.g., ROI frame 114) based on the correlation between two images to be synthesized.

As result, the probe moving speed can be reflected in the display attribute of the specific object without provision of a speed sensor or the like in ultrasound probe 2.

The invention made by the present inventor has been specifically described above based on the embodiment, but the present invention is not limited to the above embodiment and may be changed in the scope not deviating from the invention.

For example, in the embodiment, the display attribute of the specific object (e.g., ROI frame 114) has been changed when the probe moving speed is faster than the optimum speed or when the probe moving speed is slower than the optimum speed, but the display attribute of the specific object may be changed only when the probe moving speed is faster than the optimum speed, or the display attribute of the specific object may be changed only when the probe moving speed is slower than the optimum speed.

For example, in the embodiment, the display attribute of the specific object (e.g., ROI frame 114) has been changed based on the deviation between the probe moving speed and the optimum speed, but the deviation may not be reflected in the display attribute of the specific object. That is, the display attributes of the specific object may only suggest whether the probe moving speed is faster or slower than the optimum speed. In this case, the magnitude of the probe moving speed with respect to the optimum speed may be suggested with the display color of the specific object instead of the display thickness thereof.

In the embodiment, the probe moving speed has been suggested with the display color and the display thickness of the specific object (e.g., ROI frame 114), but the suggestion may be made with either one. For example, by changing the luminance of the display color in stages or changing the display thickness in stages, it is possible to suggest the deviation from the optimum speed as well as the magnitude of the probe moving speed with respect to the optimum speed. Moreover, the probe moving speed may be suggested with other display attributes (e.g., a display status (lighting, blinking, changes in display brightness and display color, etc.), and a line type (dotted line, broken line, double line, etc.).

In the embodiment, the display attribute of the specific object (ROI frame 114) has been changed with reference to attribute table T, but the display attribute of the specific object may be successively changed by computing the luminance of the display color and the display thickness in accordance with the probe moving speed.

In the embodiment, the probe moving speed has been suggested with the display attributes of ROI frame 114 by using ROI frame 114 as the specific object, but the other objects 111 to 113 may each be used as the specific object.

In the embodiment, the probe moving speed obtained from the correlation information and the display attribute of the specific object (e.g., ROI frame 114) are associated with each other in attribute table T, but the correlation information and the display attribute may be associated with each other. That is, it is not necessary to compute the probe moving speed in determining the display attributes of the specific object.

Although embodiments of the present invention have been described and illustrated in detail, the disclosed embodiments are made for purpose of illustration and example only and not limitation. The scope of the present invention should be interpreted by terms of the appended claims.

What is claimed is:

1. An ultrasound diagnostic apparatus that drives an ultrasound probe so as to transmit ultrasound toward a subject and receives a reception signal based on reflected waves reflected in the subject from the ultrasound probe, to generate and display an ultrasound image, the apparatus comprising a hardware processor that:
generates an ultrasound image from the reception signal obtained by the ultrasound probe;
stitches together a plurality of the ultrasound images to generate a panorama image, the plurality of ultrasound images being successively acquired while the ultrasound probe is moved;
causes a display to display an object indicating information necessary for image diagnosis together with the ultrasound image or the panorama image;
acquires information on a moving speed of the ultrasound probe; and
changes a display attribute of at least one of the objects in accordance with the information on the moving speed of the ultrasound probe.

2. The ultrasound diagnostic apparatus according to claim 1, wherein the hardware processor changes the display attribute based on magnitude of the moving speed of the ultrasound probe with respect to a predetermined optimum speed.

3. The ultrasound diagnostic apparatus according to claim 2, wherein the hardware processor changes the display attribute based on a deviation between the moving speed of the ultrasound probe and the optimum speed.

4. The ultrasound diagnostic apparatus according to claim 1, wherein the display attribute includes at least one of a display color, a display thickness, a display status, and a line type of the object.

5. The ultrasound diagnostic apparatus according to claim 4, wherein the hardware processor changes the display attribute in accordance with the moving speed of the ultrasound probe with reference to a display attribute table in which at least one of the display color and the display thickness is set in stages.

6. The ultrasound diagnostic apparatus according to claim 1, wherein the object is a region of interest (ROI) frame indicating a target region at the time of generating the panorama image.

7. The ultrasound diagnostic apparatus according to claim 1, wherein the hardware processor changes the display attribute based on correlation between two images to be synthesized.

8. An ultrasound diagnostic method for driving an ultrasound probe so as to transmit ultrasound toward a subject and receiving a reception signal based on reflected waves reflected in the subject from the ultrasound probe, to generate and display an ultrasound image, the method comprising:
generating an ultrasound image from the reception signal obtained by the ultrasound probe;
stitching together a plurality of the ultrasound images to generate a panorama image, the ultrasound images being successively acquired while the ultrasound probe is moved;
causing a display to display an object that indicates information necessary for image diagnosis together with the ultrasound image or the panorama image;
acquiring information on a moving speed of the ultrasound probe; and
changing a display attribute of at least one of the objects in accordance with the information on the moving speed of the ultrasound probe.

9. A non-transitory computer-readable recording medium storing a program causing a computer to execute processing, the computer being of an ultrasound diagnostic apparatus that drives an ultrasound probe so as to transmit ultrasound toward a subject and receives a reception signal based on reflected waves reflected in the subject from the ultrasound probe, to generate and display an ultrasound image, the processing to be executed by the computer, comprising:
generating an ultrasound image from the reception signal obtained by the ultrasound probe;
stitching together a plurality of the ultrasound images to generate a panorama image, the ultrasound images being successively acquired while the ultrasound probe is moved;
causing a display to display an object that indicates information necessary for image diagnosis together with the ultrasound image or the panorama image;
acquiring information on a moving speed of the ultrasound probe; and
changing a display attribute of at least one of the objects in accordance with the information on the moving speed of the ultrasound probe.
